(19) United States
(12) Patent Application Publication
(10) Pub. No.: US 2025/0031302 A1
(43) Pub. Date: Jan. 23, 2025

(54) MULTILAYER THREE-DIMENSIONAL CIRCUIT SUBSTRATE, ENDOSCOPE, AND METHOD FOR MANUFACTURING MULTILAYER THREE-DIMENSIONAL CIRCUIT SUBSTRATE

(71) Applicant: OLYMPUS MEDICAL SYSTEMS CORP., Tokyo (JP)

(72) Inventors: Takanori SEKIDO, Sagamihara-shi (JP); Kenjiro KANNO, Komagane-shi (JP)

(73) Assignee: OLYMPUS MEDICAL SYSTEMS CORP., Tokyo (JP)

(21) Appl. No.: 18/906,713

(22) Filed: Oct. 4, 2024

Related U.S. Application Data
(63) Continuation of application No. PCT/JP2022/028023, filed on Jul. 19, 2022.

(51) Int. Cl. H05K 1/02; A61B 1/00; A61B 1/05; A61B 8/12; H04N 23/50; H04N 23/54; H05K 3/22

(52) U.S. Cl. CPC H05K 1/0284; A61B 1/0011; A61B 1/051; A61B 8/12; H04N 23/54; H04N 23/555; H05K 1/0274; H05K 3/22; H05K 2201/10151

(57) ABSTRACT

A multilayer three-dimensional circuit substrate includes a first substrate, a first protective metal layer, a second protective metal layer, a second substrate, a first interlayer connecting member, and a second interlayer connecting member. The first protective metal layer has a lower absorption rate of light than a first wiring provided on a first surface of the first substrate. The second protective metal layer has a lower absorption rate of light than a second wiring provided on a second surface of the first substrate. The second substrate is configured such that a thickness between a fifth surface and a sixth surface is thinner than a thickness between a third surface and a fourth surface. A sensor is provided on the fifth surface.

### CROSS-REFERENCE TO RELATED APPLICATION

This application is a continuation application of PCT/JP2022/028023 filed on Jul. 19, 2022, the entire contents of which are incorporated herein by this reference.

### BACKGROUND OF THE INVENTION

### 1. Field of the Invention

The present disclosure relates to a multilayer three-dimensional circuit substrate including a first substrate and a second substrate formed on the first substrate, an endoscope including the multilayer three-dimensional circuit substrate, and a method for manufacturing the multilayer three-dimensional circuit substrate.

### 2. Description of the Related Art

Conventionally, a multilayer wiring substrate having a plurality of layers of conductor layers and insulating layers has been made into a product. For example, Japanese Patent Application Laid-Open Publication No. 2009-38094 discloses a method for manufacturing a multilayer wiring substrate. In the manufacturing method disclosed in the publication, a prepreg and a metal foil are stacked in sequence on an inner layer material on which an inner layer circuit is formed, to be integrated. Next, after patterning the metal foil into a hole shape, a laser is applied to create a via hole in the patterned area. Then, a base electroless plating is formed, and thereafter electrolytic plating is used to form an upper wiring layer and fill the via hole.

The publication further discloses as follows in the paragraph [0006], for example. The inner layer material is the one used for a general inner layer of a wiring substrate. As the inner layer material, in general, a resin-impregnated base material formed by impregnating a reinforcing base material with a resin composition is used. Circuits are formed on a top surface and/or a bottom surface of the required number of resin-impregnated base materials. The circuits are formed by stacking metal foils, which are formed of single, alloy, or composite foil of copper, aluminum, brass, nickel, iron, etc., to be integrated, and by performing etching and the like on the integrated metal foils.

### SUMMARY OF THE INVENTION

A multilayer three-dimensional circuit substrate according to a present embodiment includes: a first substrate, a first protective metal layer, a second protective metal layer, a second substrate, a first interlayer connecting member, and a second interlayer connecting member. The first substrate includes a first surface, a second surface, a first wiring provided on the first surface, and a second wiring provided on the second surface, and a normal of the first surface and a normal of the second surface intersect with each other on a first plane orthogonal to the first surface and the second surface. The first protective metal layer is configured to cover a first part of the first wiring, and to be electrically connected to the first wiring. The second protective metal layer is configured to cover a second part of the second wiring, and to be electrically connected to the second wiring. The second substrate includes a third surface, a fourth surface, a fifth surface, and a sixth surface, a third wiring provided on the third surface, and a fourth wiring provided on the fifth surface, and the second substrate is provided on a surface of the first substrate such that the fourth surface faces the first surface, and the sixth surface faces the second surface. The second substrate further includes a first hole and a second hole, the first hole allowing the third surface and the fourth surface to communicate with each other, and exposing a part of the first protective metal layer, the second hole allowing the fifth surface and the sixth surface to communicate with each other, and exposing a part of the second protective metal layer, a normal of the third surface and a normal of the fifth surface intersecting with each other on a second plane orthogonal to the third surface and the fifth surface. The first interlayer connecting member is provided in the first hole, and configured to electrically connect the first protective metal layer and the third wiring. The second interlayer connecting member is provided in the second hole, and configured to electrically connect the second protective metal layer and the fourth wiring. The first protective metal layer has a lower absorption rate of light in a predetermined wavelength than the first wiring. The second protective metal layer has a lower absorption rate of light in the predetermined wavelength than the second wiring. The second substrate is configured such that a thickness between the fifth surface and the sixth surface is thinner than a thickness between the third surface and the fourth surface. A sensor is provided on the fifth surface.

An endoscope according to an embodiment includes: an insertion portion configured to be inserted into a subject, and a multilayer three-dimensional circuit substrate provided in a distal end portion of the insertion portion. The multilayer three-dimensional circuit substrate includes: a first substrate, a first protective metal layer, a second protective metal layer, a second substrate, a first interlayer connecting member, and a second interlayer connecting member. The first substrate includes a first surface, a second surface, a first wiring provided on the first surface, and a second wiring provided on the second surface, a normal of the first surface and a normal of the second surface intersecting with each other on a first plane orthogonal to the first surface and the second surface. The first protective metal layer is configured to cover a first part of the first wiring, and to be electrically connected to the first wiring. The second protective metal layer is configured to cover a second part of the second wiring, and to be electrically connected to the second wiring. The second substrate includes a third surface, a fourth surface, a fifth surface, and a sixth surface, a third wiring provided on the third surface, and a fourth wiring provided on the fifth surface, and the second substrate is provided on a surface of the first substrate such that the fourth surface faces the first surface, and the sixth surface faces the second surface. The second substrate further includes a first hole and a second hole, the first hole allowing the third surface and the fourth surface to communicate with each other, and exposing a part of the first protective metal layer, the second hole allowing the fifth surface and the sixth surface to communicate with each other, and exposing a part of the second protective metal layer, a normal of the third surface and a normal of the fifth surface intersecting with each other on a second plane orthogonal to the third surface and the fifth surface. The first interlayer connecting member is provided in the first hole, and configured to electrically connect the first protective metal layer and the third wiring. The second interlayer connecting member is provided in the second hole, and configured to electrically connect the second protective metal layer and the fourth wiring. The first protective metal layer has a lower absorption rate of light in a predetermined wavelength than the first wiring. The second protective metal layer has a lower absorption rate of the light in the predetermined wavelength than the second wiring. The second substrate is configured such that a thickness between the fifth surface and the sixth surface is thinner than a thickness between the third surface and the fourth surface. A sensor is provided on the fifth surface.

A method for manufacturing a multilayer three-dimensional circuit substrate according to an embodiment includes: forming a first wiring on a first surface of a first substrate; forming a second wiring on a second surface of the first substrate, a normal of the first surface and a normal of the second surface intersecting with each other, on a first plane orthogonal to the first surface and the second surface; forming a first protective metal layer configured to cover a first part of the first wiring, and to be electrically connected to the first wiring; forming a second protective metal layer configured to cover a second part of the second wiring, and to be electrically connected to the second wiring; providing a second substrate on the first substrate, the second substrate including a third surface, fourth surface, a fifth surface, and a sixth surface, a third wiring provided on the third surface, and a fourth wiring provided on the fifth surface, the fourth surface facing the first surface, the sixth surface facing the second surface, a normal of the third surface and a normal of the fifth surface intersecting with each other on a second plane orthogonal to the third surface and the fifth surface; forming, by laser, a first hole that allows the third surface and the fourth surface to communicate with each other and exposes a part of the first protective metal layer; forming, by the laser, a second hole that allows the fifth surface and the sixth surface to communicate with each other and exposes a part of the second protective metal layer; forming a first interlayer connecting member in the first hole, the first interlayer connecting member electrically connecting the first protective metal layer and the third wiring; and forming a second interlayer connecting member in the second hole, the second interlayer connecting member electrically connecting the second protective metal layer and the fourth wiring. The first protective metal layer has a lower absorption rate of light of the laser than the first wiring, the second protective metal layer has a lower absorption rate of the light of the laser than the second wiring, the second substrate is configured such that a thickness between the fifth surface and the sixth surface is thinner than a thickness between the third surface and the fourth surface, and a sensor is provided on the fifth surface.

### BRIEF DESCRIPTION OF THE DRAWINGS

FIG. 1 is a cross-sectional view showing a configuration of a multilayer three-dimensional circuit substrate according to a first embodiment of the present disclosure.

FIG. 2 is a plan view showing a configuration in the vicinity of a first interlayer connecting member of the multilayer three-dimensional circuit substrate of the first embodiment.

FIG. 3 is a flowchart showing a manufacturing method of the multilayer three-dimensional circuit substrate according to the first embodiment.

FIG. 4 is a cross-sectional view showing a configuration of a multilayer three-dimensional circuit substrate according to a second embodiment of the present disclosure.

FIG. 5 is a cross-sectional view showing a configuration of a multilayer three-dimensional circuit substrate according to a third embodiment of the present disclosure, and is a graph showing a change in a cross-sectional area of a first substrate.

FIG. 6 is a cross-sectional view showing a configuration of a multilayer three-dimensional circuit substrate according to a fourth embodiment of the present disclosure.

FIG. 7 is a cross-sectional view showing a configuration of an image pickup unit using a multilayer three-dimensional circuit substrate according to a fifth embodiment of the present disclosure.

FIG. 8 is a perspective view showing a configuration of an endoscope system including an endoscope in which the image pickup unit of the fifth embodiment of the present disclosure is arranged.

### DETAILED DESCRIPTION OF THE PREFERRED EMBODIMENT(S)

Hereinafter, embodiments of the present disclosure will be described with reference to drawings. However, the present disclosure is not limited by the embodiments to be described below.

Note that in the description in the drawings, the same or corresponding elements are attached with the same reference signs as appropriate. In addition, the drawings are schematic, and care should be taken to the fact that a relationship among the lengths of the respective elements, a ratio of the length of a certain element to that of another element, the number of the respective elements, and the like are sometimes different from the actual ones for simplification of the description. Furthermore, the respective drawings sometimes include parts in which the relationships and ratios among the lengths of the elements, and the like are different.

### FIRST EMBODIMENT

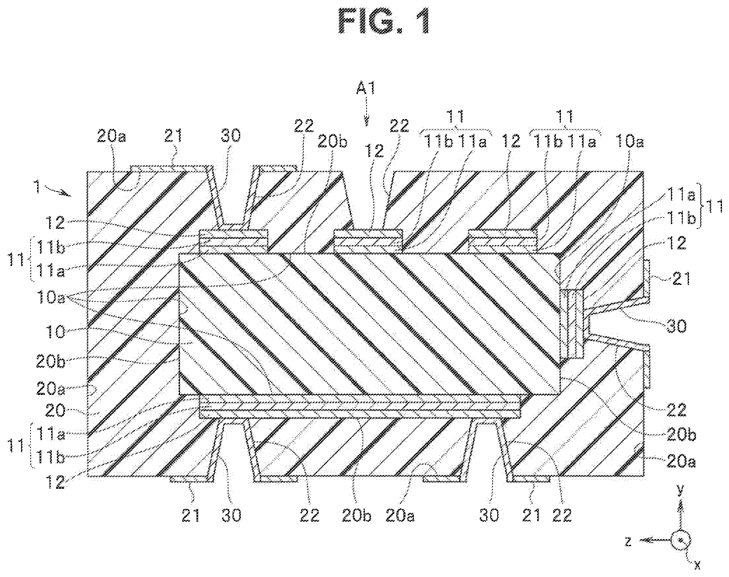

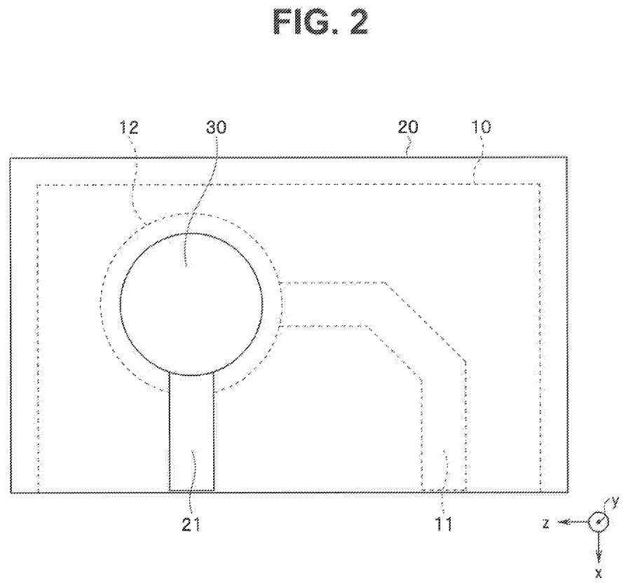

FIG. 1 to FIG. 3 show the first embodiment of the present disclosure. FIG. 1 is a cross-sectional view showing a configuration of a multilayer three-dimensional circuit substrate 1 according to a first embodiment. FIG. 2 is a plan view showing a configuration in the vicinity of a first interlayer connecting member 30 (hereinafter, also referred to as “interlayer connecting member 30”) of the multilayer three-dimensional circuit substrate 1 of the first embodiment.

The multilayer three-dimensional circuit substrate 1 includes a first substrate 10 and a second substrate 20.

The first substrate 10 includes a first surface 10a. In the cross-sectional view in FIG. 1, shows two first surfaces 10a which are parallel to the xz-plane and which differ in positions in the y direction, and two first surfaces 10a which are parallel to the xy-plane and which differ in positions in the z direction. Note that, although not shown in FIG. 1, the first substrate 10 may further include two first surfaces 10a which are parallel to the yz-plane and which differ in positions in the x direction. Among the plurality of first surfaces 10a, the two first surfaces 10a parallel to the xz-plane are principal surfaces with the largest areas on the first substrate 10.

A first wiring 11 is provided as needed on the first surfaces 10a. Specifically, FIG. 1 shows an example in which the first wiring 11 is provided on the two first surfaces 10a parallel to the xz-plane and the first surface 10a which is parallel to the xy-plane and located in the z-negative direction, and the first wiring 11 is not provided on the first surface 10a which is parallel to the xy-plane and which is located in the z-positive direction. How and on which of the plurality of first surfaces 10a the first wiring 11 is provided can be designed as appropriate.

The first wiring 11 is formed as a wiring pattern, for example. Electric components such as capacitors, and the like may be mounted to the first wiring 11.

The first wiring 11 includes copper (Cu) and nickel (Ni). Specifically, the first wiring 11 includes, on each of the first surfaces 10a, a first layer 11a formed of copper (Cu), and includes, on the first layer 11a, a second layer 11b formed of nickel (Ni).

A first protective metal layer 12 (hereinafter, referred to as “protective metal layer 12”) is formed on the first wiring 11. The protective metal layer 12 includes gold (Au). Specifically, the protective metal layer 12 is formed of gold (Au). The protective metal layer 12 has a lower absorption rate of light in a predetermined wavelength than the first wiring 11. The light in the predetermined wavelength is, for example, near-infrared (NIR: wavelength band 0.75 to 1.4 μm) light or short-wavelength infrared (SWIR: wavelength band 1.4 to 3.0 μm) light.

First holes 22 (hereinafter, referred to as “hole 22”) to be described later are formed by applying a laser light of a YAG laser, for example. The wavelength band of YAG laser (solid-state laser using yttrium, aluminum, and garnet) is 1.0 to 2.4 μm. In this wavelength band, the laser light absorption rate of gold (Au) is 0.02, that of copper (Cu) is 0.04-0.05, and that of nickel (Ni) is 0.1-0.3. The laser light absorption rate of gold (Au) is less than half of that of copper (Cu) and about 1/10 of that of nickel (Ni), and the protective metal layer 12 absorbs less laser light of the YAG laser than the first wiring 11.

Thus, the protective metal layer 12 formed of gold (Au) has the absorption rate of the light in the predetermined wavelength which is lower than that of the first wiring 11 formed of copper (Cu) and nickel (Ni). Accordingly, when the laser light is used to form the hole 22 to be described later, the protective metal layer 12 can prevent the laser light from reaching the first wiring 11, to thereby protect the first wiring 11 from being damaged.

Note that the YAG laser has been described here as an example of the laser to be used for forming the hole 22, but the laser is not limited to the YAG laser. For example, a carbon dioxide laser, which generates laser light with a longer wavelength band than the YAG laser, can be used to form the hole 22. An ultraviolet (UV) laser (for example, excimer laser) that generates laser light with a shorter wavelength band than the YAG laser may be used to form the hole 22.

As shown in FIG. 2, the protective metal layer 12 is formed on the second layer 11b so as to cover a first part of the first wiring 11. The protective metal layer 12 is electrically connected to the first wiring 11.

A second substrate 20 is provided on the first surfaces 10a of the first substrate 10 so as to cover the first substrate 10. When the electric components are mounted to the first wirings 11, the second substrate 20 covers the first substrate 10 including the electric components, for example.

The second substrate 20 includes an outside third surface 20a, and an inside fourth surface 20b. The fourth surface 20b faces the first surface 10a. Specifically, the fourth surface 20b faces to contact the first surface 10a. Therefore, the fourth surface 20b is parallel to the first surface 10a. In addition, the third surface 20a is parallel to the fourth surface 20b and the first surface 10a, for example. The third surface 20a and the fourth surface 20b, which are shown in FIG. 1, are parallel to the xz-plane or the xy-plane, for example. The third surface 20a is provided with a third wiring 21, depending on a design.

The second substrate 20 further has the holes 22 that allow the third surface 20a and the fourth surface 20b to communicate with each other. The hole 22 is formed by applying the laser light vertically to the third surface 20a. The application of the laser light ends when a part of the protective metal layer 12 is exposed. As a result, the hole 22 exposes a part of the protective metal layer 12 but does not expose the surface of the first substrate 10 other than the protective metal layer 12, as shown in the part indicated by the arrow A1 in FIG. 1. Therefore, the first substrate 10 is not damaged by the laser light.

Each of the holes 22 is provided with an interlayer connecting member 30 that electrically connects the protective metal layer 12 and the third wiring 21. Note that the part indicated by the arrow A1 in FIG. 1 shows, as a comparative example, the state before the interlayer connecting member 30 and the third wiring 21 are provided. However, in the actual product after manufacturing, the interlayer connecting member 30 and the third wiring 21 are provided.

The first substrate 10 and the second substrate 20 are formed of a resin, for example. Specifically, the first substrate 10 and the second substrate 20 may be formed using a thermosetting resin. This prevents the first substrate 10 from being damaged, for example, deformed due to the process load (heating) when forming the second substrate 20.

Here, the first substrate 10 and the second substrate 20 may be formed by using the same resin material. Such a configuration can improve the adhesion strength of the first substrate 10 and the second substrate 20. In addition, when a temperature change occurs in the multilayer three-dimensional circuit substrate 1, the amount of thermal strain at the boundary surface between the first substrate 10 and the second substrate 20 becomes substantially the same, which is less likely to generate stress in the multilayer three-dimensional circuit substrate 1. However, the first substrate 10 and the second substrate 20 may be formed of different resin materials.

In addition, the first substrate 10 may be formed of a thermosetting resin, and the second substrate 20 may be formed of a thermoplastic resin. In general, the thermoplastic resins have more material options than the thermosetting reins. For example, PEEK (Poly Ether Ether Ketone), which is currently used in image pickup systems in endoscopes, is exclusively a thermoplastic resin. Using a thermoplastic resin as a material used for forming the second substrate 20 located outside the multilayer three-dimensional circuit substrate 1 provides an increase in the design freedom.

Furthermore, the first substrate 10 may be formed of a first thermoplastic resin and the second substrate 20 may be formed of a second thermoplastic resin. In this case, the melting point Tm2 of the second thermoplastic resin may be lower than the melting point Tm1 of the first thermoplastic resin (Tm1>Tm2). This can prevent the first substrate 10 from deforming when the second substrate 20 is formed on the first substrate 10.

Note that a configuration in which the first substrate 10 is formed of a thermoplastic resin and the second substrate 20 is formed of a thermosetting resin may be employed.

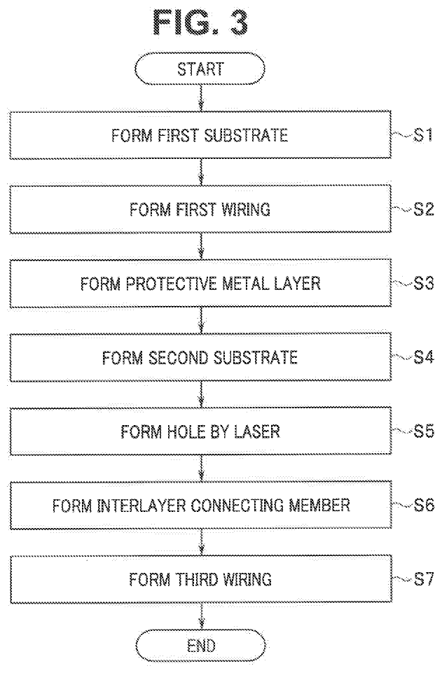

FIG. 3 is a flowchart showing a manufacturing method of the multilayer three-dimensional circuit substrate 1 according to the first embodiment. Note that FIG. 3 shows an outline of the manufacturing method of the multilayer three-dimensional circuit substrate 1.

When the processing in FIG. 3 is started, the first substrate 10 is formed of a resin (step S1). The first substrate 10 is formed by injection molding or cutting processing by using, for example, the above-mentioned PEEK (Poly Ether Ether Ketone) as the material.

Here, the PEEK which is the thermoplastic resin is shown as the example of the material for the first substrate 10, but the material is not limited to the PEEK, as described above. In addition, as the method of processing the first substrate 10, additive manufacturing using a 3D printer, and the like may be used.

Next, the first wiring 11 is formed on the first surface 10a of the first substrate 10 (step S2). As a method for forming the first wiring 11, for example, the LDS (Laser Direct Structuring) method may be used, in which a film is formed by plating only at the part where the base material is activated by applying laser light.

In addition, photolithography process may be used, and an additive process may be performed to form the film by plating only at the part corresponding to the first wiring 11. Alternatively, the photolithograph process may be used, and a subtractive process may be performed to form the film on the entire surface by plating, and then etching is performed to remove unnecessary portions other than the first wiring 11.

Note that FIG. 1 shows, as one example, the example in which the first wiring 11 is configured by stacking the second layer 11b formed of nickel (Ni) on the first layer 11a formed of copper (Cu), but the first wiring 11 is not limited to this example. The first wiring 11 may be formed of one kind of metal. In addition, the first wiring 11 may be formed by staking a plurality of other kinds of metals.

Subsequently, the protective metal layer 12, which covers the first part of the first wiring 11 and which is electrically connected to the first wiring 11, is formed (step S3). The first part of the first wiring 11 is a target part on which interlayer connection is performed.

Furthermore, the second substrate 20 is formed on the first surface 10a of the first substrate 10 (step S4). The second substrate 20 is formed by injection molding (insert molding for embedding the first substrate 10) or mold forming, liquid resin supply by dispensing, and heat curing.

After that, the hole 22 that allows the third surface 20a and the fourth surfaces 20b to communicate with each other is formed on the second substrate 20 by the laser (step S5). As described above, the application of the laser light ends when a part of the protective metal layer 12 is exposed in an appropriate size.

Then, the interlayer connecting member 30 that is electrically connected to the protective metal layer 12 is formed in the hole 22 (step S6). The interlayer connecting member 30 is formed on the surface of the hole 22 and on the protective metal layer 12 (or on the first wiring 11), for example, by the LDS method. However, the interlayer connecting member 30 may be formed by another method similarly as the first wiring 11.

Furthermore, the third wiring 21 that is electrically connected to the interlayer connecting member 30 is formed on the third surface 20a of the second substrate 20 (step S7), to end the processing. The third wiring 21 may be formed of one kind of metal, or may be formed by stacking a plurality of kinds of metals. In one example, copper (Cu), nickel (Ni), and gold (Au) are stacked in this order from the bottom layer to the top layer to form the third wiring 21.

Note that the manufacturing method shown in FIG. 3 is just one example, and the order of the processing steps is not limited to that shown in FIG. 3. For example, the order of steps S6 and S7 may be reversed. In addition, in the case where the interlayer connecting member 30 and the third wiring 21 are formed of the same material, the step S6 and the step S7 may be performed simultaneously. Furthermore, after performing the step S7, the processing in the step S5 and the step S6 may be performed in sequence or simultaneously.

According to the first embodiment, the first wiring 11 formed of copper (Cu) and nickel (Ni) is provided with the protective metal layer 12 formed of gold (Au) having the low laser light absorption rate, thus preventing a damage on the first wiring 11 when forming the hole 22 by the laser. Such a configuration can lead to an improvement in the yield when manufacturing the multilayer three-dimensional circuit substrate 1.

In addition, the hole 22 is formed by applying laser light, and thereby eliminate the limitation of the depth of the hole 22. Furthermore, after forming the hole 22, the interlayer connecting member 30 is formed by plating, to thereby enable the interlayer connecting member 30 to be formed without being limited by the depth of hole 22. Thus, the design flexibilities in the thickness of the second substrate 20, and in the hole 22 and the interlayer connecting member 30, etc., are increased.

The hole 22 is formed by applying the laser light, to thereby enable finer processing than, for example, using a mold to form the hole 22. Such a configuration enables the wiring to be formed at a higher density than in the case of using the mold, to thereby reduce the size of the multilayer three-dimensional circuit substrate 1 and allow greater flexibility in the wiring.

### Second Embodiment

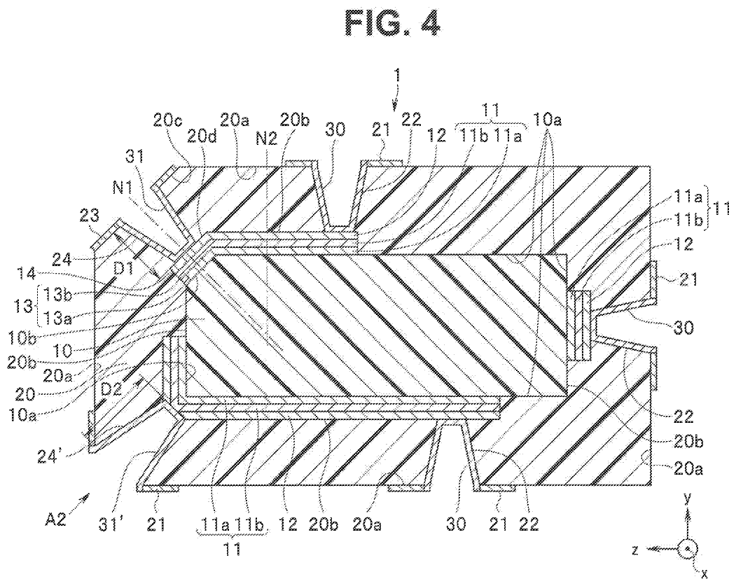

FIG. 4 is a cross-sectional view showing a configuration of a multilayer three-dimensional circuit substrate 1 according to the second embodiment of the present disclosure. In the second embodiment, the same parts as those in the first embodiment are attached with the same reference signs and descriptions thereof will be omitted as appropriate. In the second embodiment, points different from the first embodiment will be mainly described.

The first substrate 10 further includes a second surface 10b. The second surface 10b is formed as an inclined surface (chamfering part) on a ridge where the first surface 10a parallel to the xz-plane and the first surface 10a parallel to the xy-plane intersect with each other. In other words, the second surface 10b is not parallel or perpendicular to any of the xz-plane and xy-plane.

The second surface 10b is provided with a second wiring 13. The second wiring 13 includes copper (Cu) and nickel (Ni). Specifically, the second wiring 13 has a first layer 13a formed of copper (Cu) on the second surface 10b and a second layer 13b formed of nickel (Ni) on the first layer 13a. The second wiring 13 is electrically connected to the first wiring 11, for example.

A second protective metal layer 14 is formed over the second wiring 13. The second protective metal layer 14 covers a second part of the second wiring 13. The second part of the second wiring 13 is the target part on which interlayer connection is performed.

The second protective metal layer 14 is electrically connected to the second wiring 13. The second protective metal layer 14 includes gold (Au). Specifically, the second protective metal layer 14 is formed of gold (Au). Similarly as the above-described first protective metal layer 12 has the lower absorption rate of the light in the predetermined wavelength than the first wiring 11, the second protective metal layer 14 has a lower absorption rate of the light in the predetermined wavelength than the second wiring 13.

FIG. 4 shows a cross-sectional view of a multilayer three-dimensional circuit substrate 1 along a first plane (plane parallel to the yz-plane) orthogonal to the second surface 10b and the first surface 10a. On the first plane, it is assumed that the normal of the second surface 10b is N1, and the normal of the first surface 10a is N2. At this time, the normal N1 of the second surface 10b and the normal N2 of the first surface 10a intersect with each other on the first plane.

The second substrate 20 includes an outside fifth surface 20c and an inside sixth surface 20d.

The fifth surface 20c is provided with a fourth wiring 23. The fourth wiring 23 may be formed of one kind of metal, or may be formed by stacking a plurality of kinds of metals. In one example, the fourth wiring 23 is formed by stacking copper (Cu), nickel (Ni), gold (Au) in this order.

The sixth surface 20d faces the second surface 10b, specifically faces to contact the second surface 10b. Thus, the sixth surface 20d is parallel to the second surface 10b. In addition, the fifth surface 20c is parallel, for example, to the sixth surface 20d and the second surface 10b. Thus, the fifth surface 20c and sixth surface 20d are neither parallel nor perpendicular to any of the xz-plane and xy-plane, similarly as the second surface 10b.

Furthermore, the above-described first plane serves also as a second plane orthogonal to the fifth surface 20c and the third surface 20a. On the second plane, the normal of the fifth surface 20c is N1, similarly as the normal of the second surface 10b.

For example, the third surface 20a is parallel to the fourth surface 20b and the first surface 10a. In this case, the normal of the third surface 20a is N2, similarly as the normal of the first surface 10a.

In this case, the normal N1 of the fifth surface 20c and the normal N2 of the third surface 20a intersect with each other on the second plane.

Specifically, on the first plane, the normal N1 of the second surface 10b and the normal N2 of the first surface 10a intersect with each other at an acute angle or an obtuse angle. In addition, on the second plane, the normal N1 of the fifth surface 20c and the normal N2 of the third surface 20a intersect with each other at an acute angle or an obtuse angle.

The second substrate 20 further includes a second hole 24 that allows the fifth surface 20c and the sixth surface 20d to communicate with each other. The second hole 24 is formed by applying the laser light vertically to the fifth surface 20c. The application of the laser light ends when a part of the second protective metal layer 14 is exposed. As a result, the second hole 24 exposes a part of the second protective metal layer 14 but does not expose the surface of the first substrate 10 other than the second protective metal layer 14. Therefore, the first substrate 10 is not damaged by the laser light.

The second hole 24 is provided with a second interlayer connecting member 31 electrically connecting the second protective metal layer 14 and the fourth wiring 23.

Note that the part shown by the arrow A2 in FIG. 4 is an illustration part to describe a comparative example. The part shown by the arrow A2 shows the comparative example in which a second hole 24′ and a second interlayer connecting member 31′ are provided, without providing an inclined surface (second surface 10b) on a ridge where the first surface 10a parallel to the xz-plane and the first surface 10a parallel to the xy-plane intersect with each other, and without providing the fifth surface 20c and the sixth surface 20d to the second substrate 20 A.

When it is supposed that the depth of the second hole 24′ is D2 and the depth of the second hole 24 is D1, D2 is deeper than D1 (D2>D1).

In general, when the second hole and the second interlayer connecting member are provided near the corners (points at which three or more surfaces intersect one another) or near the ridges (lines on which two surfaces intersect with each other) of the rectangular-shaped first substrate 10 and second substrate 20, that is, near the end portions of the substrates, the thickness of the resin to be processed increases, compared with the case where the second hole and the second interlayer connecting member are provided on the rectangular-shaped substrate surface. In this case, if laser is used to process the resin, the amount of heat applied to the substrates increases, and the damage to the substrates caused by the heat increases.

In contrast, according to the second embodiment, the inclined surface is provided near the corners or the ridges, and then the second hole 24 and the second interlayer connecting member 31 are formed, to thereby reduce the thickness of the resin, which has to be processed, to be equal to that in the case where the second hole and the second interlayer connecting member are formed on the rectangular-shaped substrate surface. Such a configuration can reduce the heat damage to the multilayer three-dimensional circuit substrate 1 caused by the laser processing.

Furthermore, the second embodiment can provide substantially the same effects as those in the above-described first embodiment.

### Third Embodiment

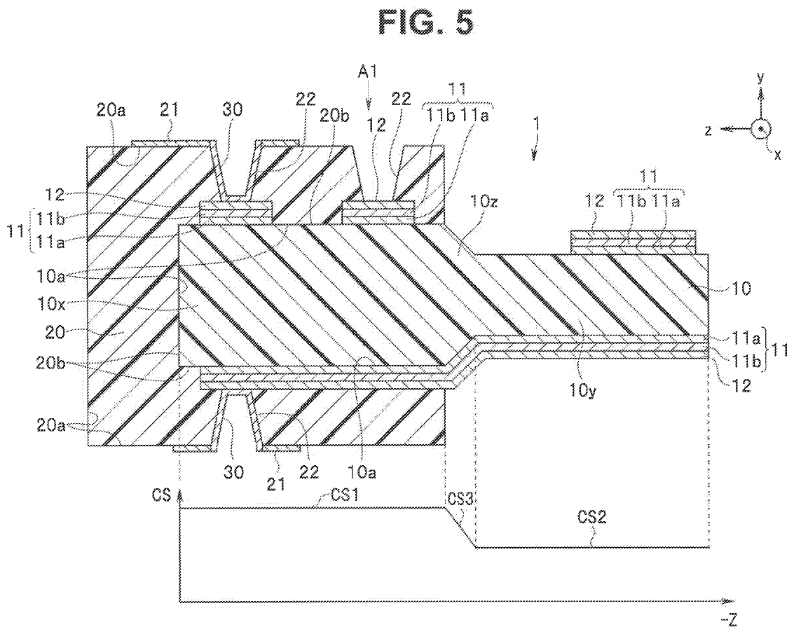

FIG. 5 is a cross-sectional view showing a configuration of a multilayer three-dimensional circuit substrate 1 according to the third embodiment of the present disclosure, and is a graph showing a change in a cross-sectional area of a first substrate 10. In the third embodiment, the same parts as those in the first and second embodiments are attached with the same reference signs and descriptions thereof will be omitted as appropriate. In the third embodiment, the points different from the first and second embodiments will be mainly described.

The first substrate 10 has a first substrate part 10x, a second substrate part 10y, and a third substrate part 10z.

The first substrate part 10x is covered with the second substrate 20. The second substrate part 10y is exposed from the second substrate 20. The third substrate part 10z connects the first substrate part 10x and the second substrate part 10y. The third substrate part 10z may be covered with the second substrate 20 or may be exposed from the second substrate 20. Alternatively, a part of the third substrate part 10z may be covered with the second substrate 20 and another part of the third substrate part 10z may be exposed from the second substrate 20.

Although the part shown by the arrow A1 in FIG. 5 shows, similarly as the part shown by the arrow A1 in FIG. 1, the state before the interlayer connecting member 30 and the third wiring 21 are provided, as a comparative example, but in the actual product after manufacturing, the interlayer connecting member 30 and the third wiring 21 are provided.

A linear line connecting the first substrate part 10x and the second substrate part 10y via the third substrate part 10z is a linear line in the z direction in the example shown in FIG. 5. The second substrate part 10y, the third substrate part 10z, and the first substrate part 10x are arranged in this order along the z direction.

It is assumed that a first cross-sectional area of the first substrate part 10x perpendicular to the linear line in the z direction is CS1, a second cross-sectional area of the second substrate part 10y is CS2, and a third cross-sectional area of the third substrate part 10z is CS3. At this time, the cross-sectional area CS of the first substrate 10 changes as shown in the graph on the lower side of FIG. 5. Note that the right direction of the horizontal axis is −z direction in the graph.

For example, the first cross-sectional area CS1 and the second cross-sectional area CS2 remain at their respective constant values, even if the position in the z direction changes, and the first cross-sectional area CS1 is larger than the second cross-sectional area CS2 (CS1>CS2). However, the cross-sectional areas are not limited to the above-described example, but the first cross-sectional area CS1 and the second cross-sectional area CS2 may be changed along the z direction.

The third cross-sectional area CS3 has no discontinuously varying points, and continuously varies and monotonically decreases according to the position in the −z direction (i.e., continuously varies and monotonically increases according to the position in the z direction). The third cross-sectional area CS3 is equal to the first cross-sectional area CS1, at a first position where the first substrate part 10x and the third substrate part 10z are connected. The third cross-sectional area CS3 is equal to the second cross-sectional area CS2, at a second position where the second substrate part 10y and the third substrate part 10z are connected. The third cross-sectional area CS3 decreases continuously from the first position toward the second position.

For example, in order to reduce the size of the multilayer three-dimensional circuit substrate 1 as much as possible, there is a case where a part of the first substrate 10 may be exposed from the second substrate 20. In such a case, the boundary between the exposed part of the first substrate 10 and the second substrate 20 is not a continuous bulk body, but a bonded portion, and a stress easily concentrates at the bonded portion. If the multilayer three-dimensional circuit substrate 1 has such a structure that the cross-sectional area of the boundary part of the first substrate 10 exposed from the second substrate 20 changes abruptly, the concentrated stress may cause destruction of the substrate, starting from the point where the cross-sectional area changes abruptly.

In contrast, the third embodiment employs a structure in which the third cross-sectional area CS3 of the third substrate part 10z continuously increases from the second substrate part 10y toward the first substrate part 10x. Such a structure can alleviate the concentration of the stress on the boundary portion of the first substrate 10 exposed from the second substrate 20 when an external force or the like acts on the multilayer three-dimensional circuit substrate 1. This prevents the first substrate 10 and second substrate 20 from being separated from each other.

Thus, the third embodiment is capable of providing substantially the same effects as those in the first and second embodiments.

### Fourth Embodiment

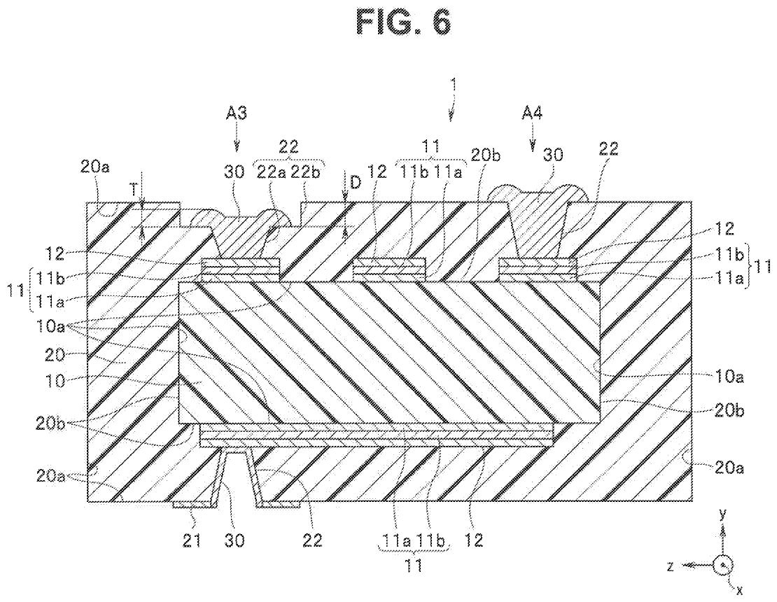

FIG. 6 is a cross-sectional view showing a configuration of a multilayer three-dimensional circuit substrate 1 according to the fourth embodiment of the present disclosure. In the fourth embodiment, the same parts as those in the first to third embodiments are attached with the same reference signs and descriptions thereof will be omitted. In the fourth embodiment, the points different from the first to third embodiments will be mainly described.

The hole 22, which is shown in the part of the arrow A3 in FIG. 6, includes a first step 22a and a second step 22b along a first direction (y direction in the example shown in FIG. 6) from the fourth surface 20b toward the third surface 20a. The cross-sectional area, which is parallel to the xz-plane, of the hole 22 i.e., the diameter of the cross-section of the hole 22 increases discontinuously at the boundary between the first step 22a and the second step 22b. Accordingly, the cross-sectional area parallel to the xz-plane is larger in the second step 22b than in the first step 22a.

The second step 22b of the hole 22 has a predetermined depth D in the y direction. Thus, the depth of the first step 22a has a value obtained by subtracting the depth D from the distance between the surface of the first protective metal layer 12 and the third surface 20a.

There is a case where a configuration for enabling electric components and cables to be directly connected to the hole 22 may be expected in order to improve mounting density and further reduce the size of the product. To this end, the first step 22a of the hole 22 is filled with the interlayer connecting member 30. This causes the interlayer connecting member 30 to function as an electrode, to thereby enable the electric components and cables to be directly connected onto the interlayer connecting member 30.

Filling of the interlayer connecting member 30 is performed by layering plating along the shape of the inner surface of the first step 22a of the hole 22. Then, at the time when the inside of the step 22a is filled with the interlayer connecting member 30, the electrode thickness (the plating thickness) around the first step 22a is thicker than the thickness of the center portion, and as a result, the periphery of the interlayer connecting member 30 is raised from the upper surface of the first step 22a.

The hole 22 shown in the part indicated by the arrow A4 shows a comparative example in which the first step 22a and the second step 22b are not provided in the hole 22. If the inside of the hole 22 having no steps is filled with the interlayer connecting member 30, the interlayer connecting member 30 is raised from the third surface 20a around the hole 22, which results in an increase in the outer shape of the multilayer three-dimensional circuit substrate 1. In view of the above, in the actual products, the configuration shown in the part of the arrow A3, to be described later, is employed, instead of the configuration shown in the part of the arrow A4.

In other words, according to the fourth embodiment shown in the part of the arrow A3, the second step 22b is formed such that the predetermined depth D of the second step 22b is equal to or larger than the maximum height T of the interlayer connecting member 30 protruding from the upper surface of the first step 22a, i.e., T≤D.

As a specific example of numerical values, if the inner diameter of the hole 22 is 50 to 300 μm, plating of 50 to 300 μm would be layered in order to fill the hole 22. The maximum height T in this case is assumed to be T=50 to 300 μm.

In this case, the predetermined depth D is 50 μm or more, and may be larger than 50 μm. Specifically, when the dimensional accuracy of the second substrate 20 is +10 to 20 μm, if a margin a somewhat larger than the dimensional accuracy is set as α=20 to 50 μm, the predetermined depth D may be set as D=T+α=70 to 350 μm. Such a configuration is capable of surely prevent the interlayer connecting member 30 from protruding from the third surface 20a.

According to the fourth embodiment, the hole 22 is provided with the first step 22a and the second step 22b in the thickness direction of the second substrate 20, and the predetermined depth D of the outside second step 22b is set to be deeper than the maximum height of the raising of the interlayer connecting member 30 with which the inside first step 22a is filled, to thereby be capable of preventing the interlayer connecting member 30 from protruding from the third surface 20a. Such a configuration prevents the protrusion of the interlayer connecting member 30 and the increase in the size of the multilayer three-dimensional circuit substrate 1.

In addition, such a configuration enables the electric components and the cables to be connected to the interlayer connecting member 30, to thereby be capable of improving the mounting density and reducing the size of the multilayer three-dimensional circuit substrate 1.

Furthermore, the fourth embodiment can provide substantially the same effects as those in the above-described first to third embodiments.

### Fifth Embodiment

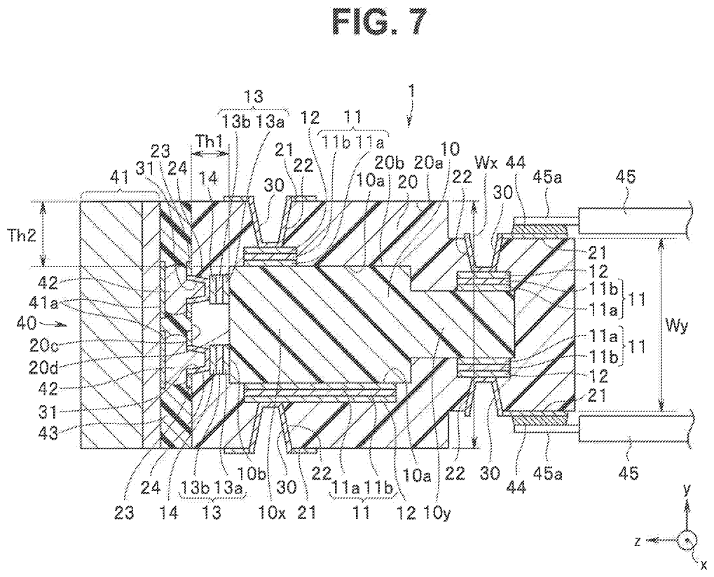

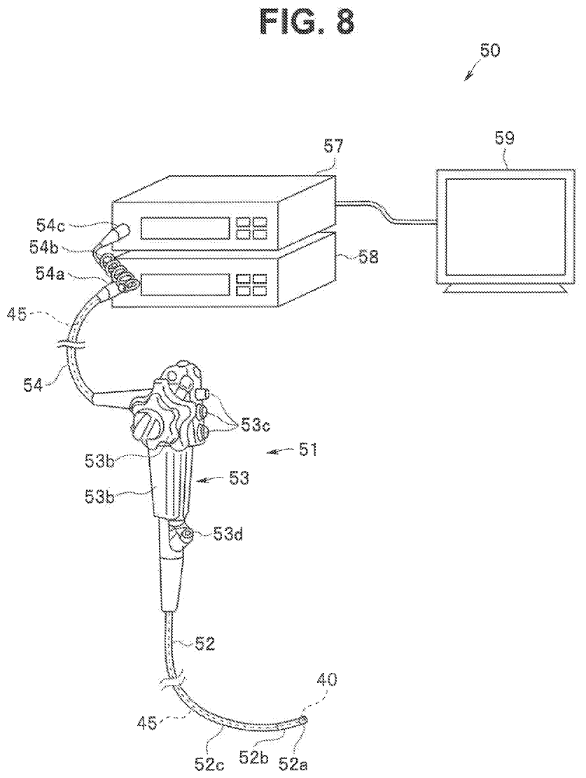

FIG. 7 and FIG. 8 show the fifth embodiment of the present disclosure. FIG. 7 is a cross-sectional view showing a configuration of the image pickup unit 40 using a multilayer three-dimensional circuit substrate 1 according to the fifth embodiment. In the fifth embodiment, the same parts as those in the first to fourth embodiments are attached with the same reference signs and descriptions thereof will be omitted. In the fifth embodiment, the points different from the first to fourth embodiments will be mainly described.

In the fifth embodiment, the surface in the z direction of the first substrate 10, which is parallel to the xy-plane of the multilayer three-dimensional circuit substrate 1 provided in the image pickup unit 40, is supposed to be a second surface 10b. However, the second surface 10b does not have to be parallel to the xy-plane.

The fifth embodiment is the same as the second embodiment in that the second substrate 20 includes the fifth surface 20c and the sixth surface 20d, the sixth surface 20d faces the second surface 10b, and the fourth wiring 23 is provided on the fifth surface 20c.

For example, the fifth surface 20c is parallel to the sixth surface 20d and the second surface 10b. In this case, the fifth surface 20c and the sixth surface 20d are parallel to the xy-plane. However, the fifth surface 20c and the sixth surface 20d do not have to be parallel to the xy-plane.

Thus, on the first plane parallel to the yz-plane, the normal of the second surface 10b and the normal of the first surface 10a intersect with each other at an acute angle, a right angle, or an obtuse angle. In addition, on the second plane parallel to the yz-plane, the normal of the fifth surface 20c and the normal of the third surface 20a intersect with each other at an acute angle, a right angle, or an obtuse angle.

The second substrate 20 is configured such that a thickness Th1 between the fifth surface 20c and the sixth surface 20d is thinner than a thickness Th2 between the third surface 20a and the fourth surface 20b. Such a configuration is achieved by mounting the electric components such as capacitors to the first wiring 11 of the first surface 10a but mounting no electric components to the second wiring 13 of the second surface 10b (as described above, the second substrate 20 is formed so as to cover the first substrate 10 including the electric components).

The second substrate 20 further has one or more second holes 24 that allows the fifth surface 20c to communicate with the sixth surface 20d. The second hole 24 is provided with a second interlayer connecting member 31 electrically connecting the second protective metal layer 14 and the fourth wiring 23. If the thickness Th1 is made thinner than the thickness Th2, the diameter of the second hole 24 can be made smaller than the diameter of the hole 22, to thereby enable a sensor 41 to be described later to be connected to the multilayer three-dimensional circuit substrate 1 at a high wiring density.

The first substrate 10 includes a first substrate part 10x and a second substrate part 10y. The thickness in the y direction of the second substrate part 10y is made thinner than the thickness in the y direction of the first substrate part 10x. Thus, a second cross-sectional area CS2 of the second substrate part 10y, which is perpendicular to the linear line in the z direction, is smaller than a first cross-sectional area CS1 of the first substrate part 10x.

Note that, in the present embodiment, not only the first substrate part 10x but also the second substrate part 10y are covered with the second substrate 20. Thus, the first substrate 10 is not provided with the third substrate part 10z, the cross-sectional area of which perpendicular to the linear line in the z direction continuously changes, as in the third embodiment.

The fifth surface 20c is configured as a surface for mounting the sensor 41, and provided with the sensor 41. Note that, in the present embodiment, the sensor 41 is an image sensor such as a CCD (charge coupled device) image sensor, a CMOS (complementary metal-oxide semiconductor) image sensor, or the like. However, the sensor 41 is not limited to these, but may be an ultrasonic probe. Furthermore, the sensor 41 may be any other appropriate sensor, such as a temperature sensor, an inertial (gyro) sensor, etc.

One or more electric contacts 41a are provided on the surface, which faces the fifth surface 20c, of the sensor 41. The above-described one or more second holes 24 are provided at positions corresponding respectively to the one or more electric contacts 41a.

The second interlayer connecting member 31 provided in the second hole 24 and the fourth wiring 23 connected to the second interlayer connecting member 31 are electrically connected respectively to corresponding electric contacts 41a using solder 42. Thus, the second interlayer connecting member 31 and the fourth wiring 23 function as electrodes to which the image sensor and the like are connected. Furthermore, a resin 43 is applied to the periphery of the solder 42 between the sensor 41 and the fifth surface 20c. This reinforces the connection of the sensor 41 and the multilayer three-dimensional circuit substrate 1.

Note that FIG. 7 shows a configuration in which the sensor 41 is connected to the surface in the z-positive direction of the multilayer three-dimensional circuit substrate 1. However, in the case where the present embodiment is applied to a lateral-viewing endoscope, for example, the sensor 41 is connected to the surface in the y-positive direction (or y-negative direction) of the multilayer three-dimensional circuit substrate 1. In addition, when the present embodiment is applied to a lateral-viewing endoscope, the inclined surface as shown in FIG. 4 of the second embodiment is provided and the sensor 41 may be connected thereto. Furthermore, FIG. 7 shows a configuration in which the sensor 41 is connected to the one surface of the multilayer three-dimensional circuit substrate 1, but the sensor may be connected to a plurality of surfaces.

The first wirings 11 and the first protective metal layers 12 that are formed on the second substrate part 10y are connected respectively to the third wirings 21 via the interlayer connecting members 30. The third wirings 21 are electrically connected to a core wire 45a of a signal cable 45 using solder 44. Thus, the third wirings 21 function as electrodes for connecting the signal cable 45.

Note that, as shown in FIG. 7, the signal cable 45 is connected to electrodes of the multilayer three-dimensional circuit substrate 1, the electrodes being provided at positions other than the part to which the sensor 41 is connected. In addition, the signal cable 45 connected to the multilayer three-dimensional circuit substrate 1 includes one or more cables, and may include a plurality of cables for practical purposes.

At this time, as described above, the thickness in the y direction of the second substrate part 10y is thinner than the thickness in the y direction of the first substrate part 10x. Thus, even if the second substrate 20 is formed with the same thickness on the first surfaces 10a of the first substrate 10, a width Wy in the y direction of the second substrate 20 formed in the second substrate part 10y is smaller than a width Wx in the y direction of the second substrate 20 formed in the first substrate part 10x.

According to such a configuration, even if the signal cables 45 are connected, the signal cables 45 do not protrude from the width Wx. This prevents the diameter of the connecting part between the image pickup unit 40 and the signal cables 45 from being larger than the diameter of the multilayer three-dimensional circuit substrate 1.

Thus, if the image pickup unit 40 having such a configuration is applied to the distal end portion 52a of the endoscope 51 (see FIG. 8), the size reduction of the distal end portion 52a of the endoscope 51 can be achieved.

Note that FIG. 7 shows an example in which the signal cables 45 are connected to the third wirings 21, but the present embodiment is not limited to this example. For example, in the multilayer three-dimensional circuit substrate 1 according to the third embodiment as shown in FIG. 5, the second substrate part 10y is exposed from the second substrate 20. In such a configuration, the signal cables 45 may be connected by solder, with the first wirings 11 (and the first protective metal layer 12) formed at the second substrate part 10y as electrodes.

Note that when only a part (the part in which electrodes for connecting the signal cables 45 are provided) of the first substrate 10 to which the signal cables 45 are connected is exposed from the second substrate 20 as shown in FIG. 5, since other parts of the first substrate 10 are covered with the second substrate 20, a solder resist that covers the surface of the substrate, to thereby insulate and protect the circuit pattern may be or may not be provided. On the other hand, the electrodes for connecting the signal cables 45 are provided on the surface of the second substrate 20 as shown in FIG. 7, a solder resist may be provided.

FIG. 8 is a perspective view showing a configuration of an endoscope system 50 including an endoscope 51 in which the image pickup unit 40 according to the fifth embodiment is disposed.

The endoscope system 50 includes the endoscope 51, an endoscope processor 57, a light source apparatus 58, and a monitor 59.

The endoscope 51 includes an insertion portion 52 configured to be inserted into a subject, an operation portion 53 provided on a proximal end side of the insertion portion 52, and a universal cord 54 extended from the operation portion 53. Note that the subject into which the insertion portion 52 is inserted may be a living organism, such as a human or an animal, or may be a non-living thing, such as a machine or building.

The insertion portion 52 includes, in the following order from the distal end side to the proximal end side, a distal end portion 52a, a bending portion 52b, and a flexible tube portion 52c.

The endoscope 51 is configured as an electronic endoscope, and the image pickup unit 40 is disposed in the distal end portion 52a. The image pickup unit 40 includes the multilayer three-dimensional circuit substrate 1 and the sensor 41 configured as an image sensor, as described above. When the insertion portion 52 is inserted into a subject, the image pickup unit 40 picks up an image inside the subject to output an image pickup signal.

The bending portion 52b is configured to be bendable, for example, in two directions, or in four directions, i.e., up, down, left, and right directions. The bending portion 52b is bent by bending operation performed through the operation portion 53. When the bending portion 52b is bent, the direction of the distal end portion 52a is changed, to thereby change the observation direction of the image pickup unit 40. In addition, the bending portion 52b is bent also for improving the insertion performance of the insertion portion 52 in the subject. Note that the endoscope 51 including the bending portion 52b is described as an example, but the endoscope 51 may be a type without the bending portion 52b.

The flexible tube portion 52c is a soft tube portion that is bent according to the shape of the subject into which the insertion portion 52 is inserted. Here, the flexible endoscope including the flexible tube portion 52c is described as an example of the endoscope 51, but the endoscope 51 may be a rigid endoscope including a rigid tube portion configured not to bend. Rigid endoscopes and flexible endoscopes in the medical fields, for example, are defined in ISO8600-1:2015.

The operation portion 53 is provided on the proximal end side of the insertion portion 52, and is a part configured to be grasped by the hand of the operator, for the operator to perform various operations related to the endoscope 51. The operation portion 53 includes a grasping portion 53a, a bending operation knob 53b, a plurality of operation buttons 53c, and a treatment instrument insertion port 53d.

The grasping portion 53a is a part for the operator to grasp the endoscope 51 with the palm.

The bending operation knob 53b is an operation device for operating bending of the bending portion 52b by using, for example, the thumb of the hand grasping the grasping portion 53a. When the bending operation knob 53b is rotated, bending wires are pulled, to thereby cause the bending portion 52b to bend.

The plurality of operation buttons 53c include a gas/liquid feeding button, a suction button, and a button related to image pickup, for example.

The gas/liquid feeding button is an operation button for feeding gas or liquid via a gas/liquid feeding channel, not shown. When the gas/liquid feeding button is operated, gas or liquid is fed from a nozzle provided at the distal end portion 52a to a cover glass provided on the distal end side of the image pickup unit 40. When the liquid is fed, the cover glass is cleaned, and when the gas is fed, the liquid adhered on the cover glass is blown off.

The suction button is an operation button to be operated for sucking a liquid, a mucosa, and the like in the subject from the distal end portion 52a side, via the treatment instrument channel also serving as a suction channel, for example.

The button related to image pickup is a button switch for performing release operation, for example.

The treatment instrument insertion port 53d is provided on the distal end side of the operation portion 53 and is an opening on the proximal end side of the treatment instrument channel. Various kinds of treatment instruments such as biopsy forceps, a high-frequency snare, and the like are inserted into the treatment instrument channel via the treatment instrument insertion port 53d. The distal end portion of the treatment instrument protrudes from the distal end side opening of the treatment instrument channel provided at the distal end portion 52a, to perform various kinds of treatment on the subject.

The universal cord 54 is a connecting cord for connecting the endoscope 51 to the light source apparatus 58 and the processor 57. A connector 54a is provided on an extension end of the universal cord 54. The universal cord 54 is connected, for example, to the light source apparatus 58 using the connector 54a.

A coil-shaped electric cable 54b is extended from the connector 54a, and a connector 54c provided at an extension end of the electric cable 54b is connected to the processor 57.

The signal cable 45 connected to the image pickup unit 40 provided in the distal end portion 52a is connected to the processor 57 and the light source apparatus 58, via the insertion portion 52, the operation portion 53, and the universal cord 54 (including the connector 54a, the electric cable 54b, and the connector 54c). The processor 57 transmits the driving signal for driving the image pickup unit 40 via the signal cable 45. The image pickup signal outputted from the image pickup unit 40 is transmitted to the processor 57 via the signal cable 45.

Note that in the case where the endoscope 51 is a laparoscope or the like, for example, and is configured such that an optical image is transmitted from the distal end of the insertion portion to the proximal end side by a relay optical system, the image pickup unit 40 may be provided on the proximal end side of the endoscope 51.

In addition, a light guide, not shown, is provided inside the insertion portion 52, the operation portion 53, and the universal cord 54 of the endoscope 51. The light guide transmits the illumination light, which is emitted from the light source apparatus 58, to an illumination optical system provided at the distal end portion 52a of the insertion portion 52. The illumination optical system applies the illumination light to the subject.

The processor 57 controls the whole endoscope system 50, performs signal processing on the image pickup signal received via the signal cable 45, generates an image signal which can be displayed, and outputs the generated image signal to the monitor 59.

The monitor 59 displays an endoscopic image based on the image signal outputted from the processor 57.

Note that the processor 57 and the light source apparatus 58 are not limited to being configured separately, but may be configured as an integrated unit. In addition, the present embodiment is not limited to the configuration in which the light source apparatus 58 emits the illumination light, but may employ a configuration in which a light-emitting device such as an LED is provided at the distal end portion 52a of the insertion portion 52 and power is supplied to the light-emitting element to cause the light-emitting element to emit illumination light.

The fifth embodiment can provide substantially the same effects as those in the above-described first to fourth embodiments, and the fifth embodiment employs the configuration in which the sensor 41 is connected to the multilayer three-dimensional circuit substrate 1 including the electric components, to thereby eliminate the need for providing a substrate on which the electric components are mounted in addition to the substrate to which the sensor 41 is connected, which results in the size reduction and cost reduction of the image pickup unit 40.

Note that, in the above-described description, description has been made on the case where the present disclosure is a multilayer three-dimensional circuit substrate, an endoscope, and a method for manufacturing the multilayer three-dimensional circuit substrate, but not limited to these. For example, the present disclosure may be a method for manufacturing an endoscope including a multilayer three-dimensional circuit substrate.

In addition, the present disclosure is not limited as-is to the above described embodiments. It is possible to embody the present disclosure by modifying the constituent elements in a range without departing from the gist of the disclosure at the practical stage. In addition, various aspects of the disclosure can be achieved by appropriately combining the plurality of constituent elements disclosed in the above-described embodiments. Some of the constituent elements may be deleted from all the constituent elements shown in the embodiment, for example. Furthermore, constituent elements over different embodiments may be combined as appropriate. It goes without saying that various modifications and applications can be implemented within a range without departing from the gist of the disclosure.

What is claimed is:

1. A multilayer three-dimensional circuit substrate comprising:
a first substrate comprising a first surface, a second surface, a first wiring provided on the first surface, and a second wiring provided on the second surface, a normal of the first surface and a normal of the second surface intersecting with each other on a first plane orthogonal to the first surface and the second surface;
a first protective metal layer configured to cover a first part of the first wiring, and to be electrically connected to the first wiring;
a second protective metal layer configured to cover a second part of the second wiring, and to be electrically connected to the second wiring;
a second substrate comprising a third surface, a fourth surface, a fifth surface, and a sixth surface, a third wiring provided on the third surface, and a fourth wiring provided on the fifth surface, the second substrate being provided on a surface of the first substrate such that the fourth surface faces the first surface and the sixth surface faces the second surface, the second substrate further comprising a first hole and a second hole, the first hole allowing the third surface and the fourth surface to communicate with each other, and exposing a part of the first protective metal layer, the second hole allowing the fifth surface and the sixth surface to communicate with each other, and exposing a part of the second protective metal layer, a normal of the third surface and a normal of the fifth surface intersecting with each other on a second plane orthogonal to the third surface and the fifth surface;
a first interlayer connecting member provided in the first hole, and configured to electrically connect the first protective metal layer and the third wiring; and
a second interlayer connecting member provided in the second hole, and configured to electrically connect the second protective metal layer and the fourth wiring;
wherein the first protective metal layer has a lower absorption rate of light in a predetermined wavelength than the first wiring,
the second protective metal layer has a lower absorption rate of light in the predetermined wavelength than the second wiring,
the second substrate is configured such that a thickness between the fifth surface and the sixth surface is thinner than a thickness between the third surface and the fourth surface, and
a sensor is provided on the fifth surface.

2. The multilayer three-dimensional circuit substrate according to claim 1, wherein the light in the predetermined wavelength is near-infrared light or short-wavelength infrared light.

3. The multilayer three-dimensional circuit substrate according to claim 1, wherein the first protective metal layer includes gold.

4. The multilayer three-dimensional circuit substrate according to claim 3, wherein the first wiring includes copper and nickel.

5. The multilayer three-dimensional circuit substrate according to claim 1, wherein the first substrate is formed of a thermosetting resin.

6. The multilayer three-dimensional circuit substrate according to claim 5, wherein the second substrate is formed of a thermosetting resin.

7. The multilayer three-dimensional circuit substrate according to claim 5, wherein the second substrate is formed of a thermoplastic resin.

8. The multilayer three-dimensional circuit substrate according to claim 1, wherein
the first substrate is formed of a first thermoplastic resin,
the second substrate is formed of a second thermoplastic resin, and
a melting point of the second thermoplastic resin is lower than a melting point of the first thermoplastic resin.

9. The multilayer three-dimensional circuit substrate according to claim 1, wherein
the first wiring and the second wiring are electrically connected to each other,
the normal of the second surface and the normal of the first surface intersect with each other at an acute angle or an obtuse angle on the first plane, and
the normal of the fifth surface and the normal of the third surface intersect with each other at an acute angle or an obtuse angle on the second plane.

10. The multilayer three-dimensional circuit substrate according to claim 1, wherein the sensor is an image sensor.

11. The multilayer three-dimensional circuit substrate according to claim 1, wherein
the first substrate includes a first substrate part, a second substrate part, and a third substrate part,
the first substrate part is covered with the second substrate,
the second substrate part is exposed from the second substrate, and
the third substrate part connects the first substrate part and the second substrate part,
wherein a first cross-sectional area of the first substrate part, a second cross-sectional area of the second substrate part, and a third cross-sectional area of the third substrate part are perpendicular to a linear line connecting the first substrate part and the second substrate part via the third substrate part, and configured such that:
the first cross-sectional area is larger than the second cross-sectional area,
the third cross-sectional area is equal to the first cross-sectional area, at a first position where the first substrate part and the third substrate part are connected,
the third cross-sectional area is equal to the second cross-sectional area, at a second position where the second substrate part and the third substrate part are connected, and
the third cross-sectional area continuously decreases from the first position toward the second position.

12. The multilayer three-dimensional circuit substrate according to claim 1, wherein
the first hole includes a first step and a second step along a first direction from the fourth surface toward the third surface,
the second step of the first hole has a predetermined depth, and
the first step of the first hole is filled with the first interlayer connecting member, and the first interlayer connecting member does not protrude from the third surface.

13. The multilayer three-dimensional circuit substrate according to claim 12, wherein the predetermined depth is larger than 50 μm.

14. An endoscope comprising:
an insertion portion configured to be inserted into a subject, and a multilayer three-dimensional circuit substrate provided in a distal end portion of the insertion portion,
the multilayer three-dimensional circuit substrate comprising:
a first substrate comprising a first surface, a second surface, a first wiring provided on the first surface, and a second wiring provided on the second surface, a normal of the first surface and a normal of the second surface intersecting with each other on a first plane orthogonal to the first surface and the second surface;
a first protective metal layer configured to cover a first part of the first wiring, and to be electrically connected to the first wiring;
a second protective metal layer configured to cover a second part of the second wiring, and to be electrically connected to the second wiring;
a second substrate comprising a third surface, a fourth surface, a fifth surface, and a sixth surface, a third wiring provided on the third surface, and a fourth wiring provided on the fifth surface, the second substrate being provided on a surface of the first substrate such that the fourth surface faces the first surface and the sixth surface faces the second surface, the second substrate further comprising a first hole and a second hole, the first hole allowing the third surface and the fourth surface to communicate with each other, and exposing a part of the first protective metal layer, the second hole allowing the fifth surface and the sixth surface to communicate with each other, and exposing a part of the second protective metal layer, a normal of the third surface and a normal of the fifth surface intersecting with each other on a second plane orthogonal to the third surface and the fifth surface;
a first interlayer connecting member provided in the first hole, and configured to electrically connect the first protective metal layer and the third wiring; and
a second interlayer connecting member provided in the second hole, and configured to electrically connect the second protective metal layer and the fourth wiring,
wherein the first protective metal layer has a lower absorption rate of light in a predetermined wavelength than the first wiring,
the second protective metal layer has a lower absorption rate of the light in the predetermined wavelength than the second wiring,
the second substrate is configured such that a thickness between the fifth surface and the sixth surface is thinner than a thickness between the third surface and the fourth surface, and
a sensor is provided on the fifth surface.

15. The endoscope according to claim 14, wherein the sensor is an image sensor.

16. The endoscope according to claim 14, wherein the sensor is an ultrasonic probe.

17. A method for manufacturing a multilayer three-dimensional circuit substrate, the method comprising:
forming a first wiring on a first surface of a first substrate;
forming a second wiring on a second surface of the first substrate, a normal of the first surface and a normal of the second surface intersecting with each other, on a first plane orthogonal to the first surface and the second surface;
forming a first protective metal layer configured to cover a first part of the first wiring, and to be electrically connected to the first wiring;
forming a second protective metal layer configured to cover a second part of the second wiring, and to be electrically connected to the second wiring;
providing a second substrate on the first substrate, the second substrate comprising a third surface, fourth surface, a fifth surface, and a sixth surface, a third wiring provided on the third surface, and a fourth wiring provided on the fifth surface, the fourth surface facing the first surface, the sixth surface facing the second surface, a normal of the third surface and a normal of the fifth surface intersecting with each other on a second plane orthogonal to the third surface and the fifth surface;
forming, by laser, a first hole that allows the third surface and the fourth surface to communicate with each other and exposes a part of the first protective metal layer;
forming, by the laser, a second hole that allows the fifth surface and the sixth surface to communicate with each other and exposes a part of the second protective metal layer;
forming a first interlayer connecting member in the first hole, the first interlayer connecting member electrically connecting the first protective metal layer and the third wiring; and
forming a second interlayer connecting member in the second hole, the second interlayer connecting member electrically connecting the second protective metal layer and the fourth wiring,
wherein the first protective metal layer has a lower absorption rate of light of the laser than the first wiring,
the second protective metal layer has a lower absorption rate of the light of the laser than the second wiring,
the second substrate is configured such that a thickness between the fifth surface and the sixth surface is thinner than a thickness between the third surface and the fourth surface, and
a sensor is provided on the fifth surface.
